(19) United States
(12) Patent Application Publication
(10) Pub. No.: US 2024/0412358 A1
(43) Pub. Date: Dec. 12, 2024

(54) MEDICAL IMAGE ANALYSIS METHOD

(71) Applicant: VUNO Inc., Seoul (KR)

(72) Inventors: Kyungdoc KIM, Seoul (KR); Hongseok LEE, Seoul (KR); Chanmin PARK, Seoul (KR); Minwoo CHOO, Seoul (KR); Sangkeun KIM, Seoul (KR)

(21) Appl. No.: 18/702,737

(22) PCT Filed: Oct. 19, 2022
(86) PCT No.: PCT/KR2022/015917

§ 371 Date: Apr. 18, 2024

(30) Foreign Application Priority Data
Oct. 20, 2021 (KR) 10-2021-0139898

(51) Int. Cl. G06T 7/00; G16H 30/20; G16H 30/40; G16H 40/67

(52) U.S. Cl. CPC G06T 7/0012; G16H 30/20; G16H 30/40; G16H 40/67; G06T 2200/24; G06T 2207/20016; G06T 2207/20104; G06T 2207/30024

(57) ABSTRACT

Disclosed is a method for analyzing a medical image performed by a user terminal performed by a computing device. The method may include: determining a region of interest of a medical image based on a first user input on a user interface; transmitting identification information and contour information of the region of interest to a server, and receiving position information and staining information of a stained cell present in the region of interest from the server; distinguishing the cell present in the region of interest as positive or negative based on a threshold which is adjustable according to a second user input on the user interface by using the position information and the staining information of the stained cell present in the region of interest; and outputting analysis information generated based on the cell distinguished as positive or negative through the user interface.

### BACKGROUND

### Technical Field

The present disclosure relates to a method for analyzing a medical image, and more particularly, to a method for performing analysis on a stained cell present in a medical image, and visualizing an analysis result.

### Description of the Related Art

Conventional pathological diagnostic tests involve making cell or tissue samples collected from the human body as glass slides and examining the cell or tissue samples with a microscope. Since the conventional testing method detects and classifies each cell on the slide with the naked eyes, a testing speed is slow and it inevitably takes a long time to confirm a final test result. As a result, the conventional testing methods cause delays in diagnosis and treatment of patients. In particular, in the current era where the number of pathological diagnoses has rapidly increased due to the aging population and increase in cancer patients, the conventional testing methods are no longer considered appropriate. Therefore, the need for digital pathology has been increasing recently.

The digital pathology refers to a method of acquiring digital images from glass slides using a scanner and managing, sharing, and analyzing the digital images in a computing environment, rather than a conventional method of examining pathological diagnoses through glass slides with the naked eye through a microscope. The digital pathology provides an environment in which pathological diagnosis work can be performed efficiently by automatically analyzing the digital images of the glass slides in the computing environment. In other words, the digital pathology improves the problem of delay in test, which is a problem of the conventional testing methods, and provides an environment in which patient diagnosis and treatment can be carried out efficiently.

The digital images handled in the digital pathology are large data, ranging from hundreds of megabytes to gigabytes. Therefore, in digital pathology based on data communication between computing devices, one of the important issues is how to efficiently transmit and receive data including analysis of digital images and analysis results of digital images. For example, when loading the digital images or image analysis results from the computing device for digital pathology, network latency problems, memory problems, etc., may occur due to the high volume of data. In other words, in the digital pathology, how to effectively process the high volume of data, such as digital images acquired by scanning the glass slides, by minimizing resources (e.g., analysis, transmission, reception, etc.) is one of the important issues.

Korean Patent Unexamined Publication No. 10-2020-0117222 (Oct. 14, 2020) discloses an apparatus and a method for supporting a pathological diagnosis.

### BRIEF SUMMARY

### Technical Problem

The present disclosure is contrived in response to the background art, and has been made in an effort to perform an analysis on a stained cell present in a medical image based on efficient data processing, and visualize an analysis result.

### Technical Solution

According to an embodiment of the present disclosure for achieving the object, disclosed is a method for analyzing a medical image performed by a user terminal. The method may include: determining a region of interest of a medical image based on a first user input on a user interface; transmitting identification information and contour information of the region of interest to a server, and receiving position information and staining information of stained cells present in the region of interest from the server; distinguishing cells present in the region of interest as positive or negative based on a threshold which is adjustable according to a second user input on the user interface by using the position information and the staining information of the stained cells present in the region of interest; and outputting analysis information for the cells distinguished as positive or negative through the user interface.

In an alternative embodiment, the method may further include: converting and storing a medical image to be analyzed for each resolution level; and outputting a medical image of a resolution level corresponding to a magnification level adjusted according to a third user input on the user interface through the user interface, in the stored medical image.

In an alternative embodiment, the determining of the region of interest of the medical image based on the first user input on the user interface may include generating the region of interest of the medical image as a closed contour according to the first user input.

In an alternative embodiment, the contour information of the region of interest may include a coordinate value of a point which becomes a reference of a contour of the region of interest. In addition, the point which becomes the reference of the contour of the region of interest may be determined according to the first user input.

In an alternative embodiment, the position information of the stained cells present in the region of interest may include a coordinate value of the stained cell present in the region of interest, which is calculated from an input image including the region of interest through a neural network model of the server. In addition, the staining information of the stained cell present in the region of interest may include a staining ratio of the stained cell, which is calculated in a cell region of the region of interest corresponding to the coordinate value by the server.

In an alternative embodiment, staining ratio of the stained cells may be a ratio of an area of a positive region of cells expressed by staining in the cell region of the region of interest corresponding to the coordinate value to a total area of the cell region.

In an alternative embodiment, the staining ratio of the stained cells may be calculated based on a binary image for the cell region generated based on a staining intensity of the region of interest by the server.

In an alternative embodiment, the threshold adjustable according to the second user input on the user interface may include a first reference value indicating a staining expression intensity for distinguishing the cells present in the region of interest into positive or negative.

In an alternative embodiment, the threshold adjustable according to the second user input on the user interface may further include a second reference value for distinguishing positive cells determined according to the first reference value according to a staining expression intensity.

In an alternative embodiment, the distinguishing of the cells present in the region of interest as positive or negative may include comparing the threshold and the staining information of the stained cells present in the region of interest and distinguishing the cells as positive or negative.

In an alternative embodiment, the comparing of the threshold and the staining information of the stained cells present in the region of interest and distinguishing of the cells as positive or negative may include comparing the first reference value included in the threshold and the staining information of the stained cells present in the region of interest and distinguishing the cells as positive or negative, and comparing the second reference value included in the threshold and the staining information of the positive cells distinguished according to the first reference value, and distinguishing the positive cells according to the staining expression intensity.

In an alternative embodiment, the distinguishing of the cells present in the region of interest as positive or negative may include updating, when the threshold is adjusted by the second user input on the user interface, a result of distinguishing the cells as positive or negative based on the adjusted threshold.

In an alternative embodiment, the outputting of the analysis information through the user interface may include outputting a medical image in which positive cells and negative cells distinguished by using the first reference value included in the threshold are displayed in different colors to a first region of the user interface. In this case, the positive cells distinguished by using the first reference value included in the threshold may be displayed in different colors according to the staining expression intensity distinguished by using the second reference value included in the threshold.

In an alternative embodiment, the outputting of the medical image in which positive cells and negative cells distinguished by using the threshold are displayed in different colors to the first region of the user interface may include storing a region of interest of the medical image in which the positive cells and negative cells distinguished by using the threshold are displayed in different colors as a separate image, and converting, when the magnification level for the region of interest is adjusted or the region of interest is moved according to a third user input on the user interface, a size or a coordinate of the region of interest stored as the separate image according to the third user input.

In an alternative embodiment, the outputting of the analysis information through the user interface may include outputting, to a second region of the user interface, at least one of the number of negative cells distinguished by using the threshold, the number of positive cells distinguished by using the threshold, a first ratio based on the number of positive cells, or a second ratio based on an overall average of staining expression intensities of the stained cells present in the region of interest.

In an alternative embodiment, the user interface may include a first region receiving the first user input, and outputting a medical image for determining the region of interest, a medical image in which the region of interest is visualized, or the medical image in which the positive cells and negative cells distinguished by using the threshold displayed in different colors, a second region outputting at least one of the number of negative cells distinguished by using the threshold, the number of positive cells distinguished by using the threshold, the first ratio based on the number of positive cells, or the second ratio based on the overall average of staining expression intensities of the stained cells present in the region of interest, and a third region receiving the second user input, and outputting information on the threshold.

According to an embodiment of the present disclosure for achieving the object, disclosed is a computer program stored in a computer-readable storage medium. When the computer program is executed by one or more processors, the computer program allows the one or more processors to execute the following operations for analyzing a medical image, and the operations may include: an operation of determining a region of interest of a medical image based on a first user input on a user interface; an operation of distinguishing cells present in the region of interest as positive or negative based on a threshold which is adjustable according to a second user input on the user interface by using the position information and the staining information of the stained cells present in the region of interest; and an operation of outputting analysis information for the cells distinguished as positive or negative through the user interface. In this case, the position information and the staining information of the stained cells present in the region of interest may be received from a server receiving identification information and contour information of the region of interest.

According to an embodiment of the present disclosure for achieving the object, disclosed is a user terminal for analyzing a medical image. The user terminal may include: a processor including one or more cores; a memory; a network unit for communication with a server; and an output unit providing a user interface. In this case, the processor may be configured to determine a region of interest of a medical image based on a first user input on a user interface, distinguish cells present in the region of interest as positive or negative based on a threshold which is adjustable according to a second user input on the user interface by using the position information and the staining information of the stained cells present in the region of interest, and output analysis information for the cells distinguished as positive or negative through the user interface. Further, the network unit may transmit identification information and contour information of the region of interest to the server, and receive position information and staining information of stained cells present in the region of interest from the server.

### Advantageous Effects

According to the present disclosure, a method for performing an analysis on a stained cell present in a medical image based on efficient data processing, and visualizing an analysis result can be provided.

### BRIEF DESCRIPTION OF THE SEVERAL VIEWS OF THE DRAWINGS

FIG. 1 is a block diagram of a user terminal for analyzing a medical image according to an embodiment of the present disclosure.

FIG. 2 is a block diagram illustrating a process for analyzing a medical image based on communication between a user terminal and a server according to an embodiment of the present disclosure.

FIG. 3 is a flowchart illustrating a process of analyzing a medical image of a user terminal according to an embodiment of the present disclosure.

FIGS. 4 to 6 are conceptual views exemplarily illustrating a user interface according to an embodiment of the present disclosure.

FIGS. 7 to 9 are conceptual views exemplarily illustrating a user interface according to an additional embodiment of the present disclosure.

FIG. 10 is a flowchart illustrating an operation process of a server according to an embodiment of the present disclosure.

FIG. 11 is a conceptual view of a process of generating a binary image of a server according to an embodiment of the present disclosure.

FIG. 12 is a schematic view of a computing environment according to an embodiment of the present disclosure.

### DETAILED DESCRIPTION

Various exemplary embodiments will now be described with reference to drawings. In the present specification, various descriptions are presented to provide appreciation of the present disclosure. However, it is apparent that the exemplary embodiments can be executed without the specific description.

“Component,” “module,” “system,” and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or.” That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A,” “a case including only B,” and “a case in which A and B are combined.”

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein.

Meanwhile, the term “image” or “video” used throughout the detailed description and claims of the present disclosure refers to multi-dimensional data constituted by discrete image elements (e.g., pixels in a 2D image), and in other words, refers to an object which may be seen visually (e.g., displayed on a video screen) or a digital representation of the object (such as a file corresponding to a pixel output of CT, MRI detector, etc.).

For example, the “image” or “video” may be a medical image of a subject collected by computed tomography (CT), magnetic resonance imaging (MRI), ultrasonic waves, pathology scan, or any other medical imaging system known in the technical field of the present disclosure.

Throughout the detailed description and claims of the present disclosure, a ‘Digital Imaging and Communications in Medicine (DICOM)’ standard is a term which collectively refers to several standards used for digital image representation and communication in a medical device, so that the DICOM standard is announced by the Federation Committee, constituted in the American College Radiology (ACR) and the National Electrical Manufacturers Association (NEMA).

Further, throughout the detailed description and claims of the present disclosure, a ‘Picture Archiving and Communication System (PACS)’ is a term that refers to a system for performing storing, processing, and transmitting according to the DICOM standard, and medical images acquired by using digital medical image equipment such as X-ray, CT, MRI, and a pathology scanner may be stored in a DICOM format and transmitted to terminals inside or outside a hospital through a network, and additionally include a reading result and a medical chart.

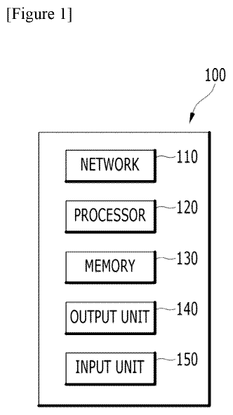

FIG. 1 is a block diagram of a user terminal for analyzing a medical image according to an embodiment of the present disclosure.

A configuration of a user terminal 100 illustrated in FIG. I is only an example shown through simplification. In an embodiment of the present disclosure, the user terminal 100 may include other components for performing a computing configuration of the user terminal 100 and only some of the disclosed components may constitute the user terminal 100.

The user terminal 100 according to an embodiment of the present disclosure may include a network unit 110, a processor 120, a memory 130, an output unit 140, and an input unit 150.

The user terminal 100 according to an embodiment of the present disclosure may include any type of terminal that receives data resources generated by an arbitrary server and performs information processing. For example, the user terminal 100 may include a personal computer (PC), a note book, a mobile terminal, a smart phone, a tablet PC, and the like and may include all types of terminals which are accessible to a wired/wireless network.

The network unit 110 according to an embodiment of the present disclosure may use an arbitrary type of wired/wireless communication system.

The network unit 110 may receive a medical image in which a tissue or cell is expressed from a medical image storage and transmission system. For example, the medical image in which the tissue or cell is expressed may be a pathology slide image including at least one tissue or cell. At this time, the pathology slide image may be understood as a scan image acquired from a glass slide through a scanner for pathology diagnosis and stored in the medical image storage and transmission system. The tissue or cell expressed in the pathology slide image may be a tissue or cell stained through immunohistochemistry.

The immunohistochemistry refers to a method for detecting a target protein or antigen in the tissue. Specifically, the immunohistochemistry refers to a method of exposing a labeled antibody that can bind to the epitope of the target protein or antigen to a tissue section, and visualizing the labeled antibody through tissue staining. For example, the immunohistochemistry may be performed by performing an antibody reaction against a cancer cell proliferation marker such as ki-67 on a glass slide, and then visualizing, through staining, a location where ki-67 is expressed using a specific solution such as Diaminobenzidine (DAB). Cells stained by using the DAB solution may be marked with a brown color in the pathology slide image. At this time, the degree of ki-67 expression in the stained cells may be distinguished depending on the intensity of the brown color. Since the ki-67 and the DAB solution are only examples used in the immunohistochemistry, the immunohistochemistry described in the present disclosure is not limited to the above-described examples.

In addition, the network unit 110 may transmit and receive arbitrary information related to medical images through communication with other terminals or servers. For example, the network unit 110 may transmit information about a region of interest of the medical image processed by the processor 120 to the server. In this case, the region of interest may be understood as a partial region of the medical image which a user wants to analyze. After the server performs analysis based on information about the region of interest of the medical image, the network unit 110 may receive analysis information about the region of interest processed by the server from the server. That is, the network unit 110 may transmit and receive data related to medical images processed by each of the user terminal and the server through communication with the server.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an exemplary embodiment of the present disclosure, processors of a plurality of computing devices may be used together to process the learning of the network function and the data classification using the network function. Further, the computer program executed in the computing device according to an exemplary embodiment of the present disclosure may be a CPU, GPGPU, or TPU executable program.

The processor 120 may generate a user interface for interaction with a user who wants to analyze the medical image. The user interface generated by the processor 120 is a medium for communication between the user and the computer, and may include various types of user interfaces such as a speech recognition-based conversational user interface (CUI), a user motion-based gesture user interface, etc., in addition to a graphic user interface (GUI). The processor 120 may recognize information input by the user related to analysis of the medical image through the user interface, and perform and control calculation operations for analyzing the medical image. Additionally, the processor 120 may visualize information derived as a result of calculations for analyzing the medical image and deliver the information to the user through the user interface. That is, the processor 120 may perform a calculation operation by reflecting an intention of the user through the user interface. Further, the processor 120 may visualize information desired by the user and deliver the visualized information to the user through the user interface.

The processor 120 may determine the region of interest of the medical image based on a user input on the user interface. The user input may be understood as a term referring to any type of information, signal, or data delivered from the user to the processor 120 through the user interface. The processor 120 may determine the region of interest in the medical image according to the user input and extract information on a region corresponding to the region of interest from the medical image. At this time, the region of interest may be a region of a closed contour having a circular or polygonal shape depending on the user input. For example, the processor 120 may determine the region of interest of the closed contour connecting points in the medical image according to a user input that specifies an arbitrary point in the medical image. When four points are specified in the medical image according to the user input on the user interface, the processor 120 may generate a closed contour connecting four points in the medical image and determine a rectangular region defined by the closed contour as the region of interest.

The processor 120 may determine whether cells present in the region of interest are positive or negative based on information on the cells present in the region of interest analyzed by the server. When the region of interest is determined as described above, the processor 120 may receive information on stained cells present in the region of interest analyzed by the server from the network unit 110. The processor 120 may distinguish the stained cells present in the region of interest as positive or negative using a threshold determined by the user input on the user interface based on the information on the stained cells present in the region of interest. The processor 120 may determine whether the cells present in the region of interest are positive cells or negative cells by comparing the information on stained cells in the region of interest with the threshold based on the user input. At this time, the threshold is a reference value indicating a staining expression intensity of the positive cells and may be adjusted through the user interface. For example, the processor 120 may compare the staining information of the stained cells analyzed through the neural network model of the server with the threshold according to the user input and determine that stained cells that are equal to or more than a first reference value included in the threshold are the positive cells. The processor 120 may compare the staining information of the stained cells analyzed through the neural network model of the server with the threshold according to the user input and determine that stained cells that are equal to or less than the first reference value included in the threshold are the negative cells. Further, the processor 120 may compare the staining information of the positive cells determined through the first reference value with at least one second reference value included in the threshold to distinguish the positive cells according to the staining expression intensity. That is, the processor 120 may determine the stained cells as negative cells or positive cells by comparing the staining information of the stained cells present in the region of interest and the threshold, and classify the positive cells into a plurality of classes according to the staining expression intensity.

The processor 120 may generate analysis information based on a result of distinguishing whether cells present in the region of interest are positive or negative. The analysis information may variously include information which may be used for pathological diagnosis based on the region of interest, such as the number of positive cells in the region of interest, the number of negative cells in the region of interest, or a ratio of positive cells in the region of interest. For example, the analysis information may include at least one of the number of negative cells in the region of interest, the number of positive cells in the region of interest, a first ratio based on the number of positive cells present in the region of interest, or a second ratio based on an overall average of staining expression intensities of the stained cells present in the region of interest. At this time, the first ratio may be understood as a ratio of the number of positive cells to the total number of stained cells present in the region of interest. Further, the second ratio may be understood as a ratio of the sum of the staining expression intensity of positive cells to the total number of staining cells present in the region of interest. The processor 120 may visualize the analysis information and output the visualized analysis information through the user interface. The processor 120 may convert the analysis information into various output formats, such as text and images, in accordance with the user interface. The processor 120 may provide the analysis information converted into various output formats to the user through the user interface.

According to an exemplary embodiment of the present disclosure, the memory 130 may store any type of information generated or determined by the processor 110 and any type of information received by the network unit 150.

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

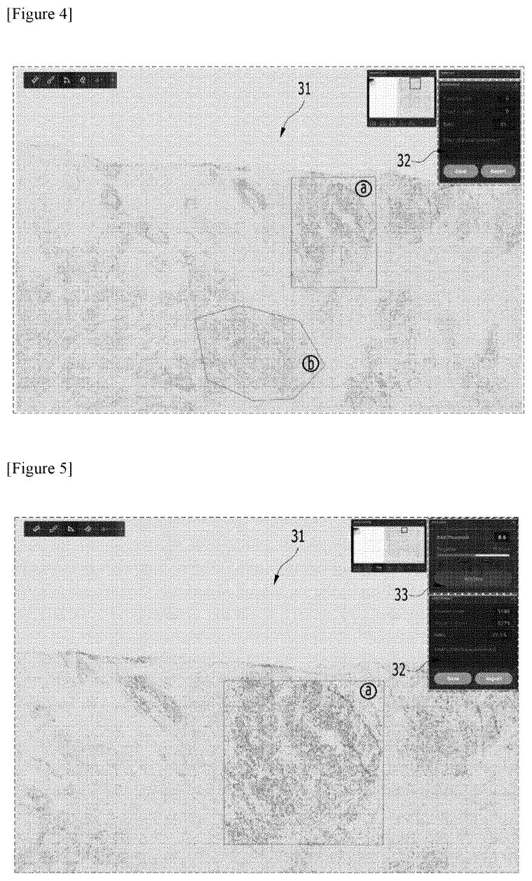

According to an embodiment of the present disclosure, the output unit 140 may display the user interface according to an embodiment of the present disclosure. The output unit 140 may display the user interface illustrated in FIGS. 4 to 6. The user interfaces shown and described below are examples only, and the present disclosure is not limited thereto.

The output unit 140 according to an embodiment of the present disclosure may output any type of information generated or determined by the processor 120 or any type of information received by the network unit 110.

The output unit 140 according to an embodiments of the present disclosure may include at least one of a liquid crystal display (LCD), a thin film transistor-liquid crystal display (TFT LCD), an organic light-emitting diode (OLED), a flexible display, and a 3D display. Some display modules among them may be configured as a transparent or light transmissive type to view the outside through the displays. This may be called a transparent display module and a representative example of the transparent display module includes a transparent OLED (TOLED), and the like.

User input may be received through the input unit 150 according to an embodiment of the present disclosure. The input unit 150 according to an embodiment of the present disclosure may include keys and/or buttons for receiving the user input. A computer program for providing the user interface according to embodiments of the present disclosure may be executed according to the user input through the input unit 150.

The input unit 150 according to embodiments of the present disclosure receives a signal by sensing a button operation or a touch input of the user or receives speech or a motion of the user through a camera or a microphone to convert the received signal, speech, or motion into an input signal. To this end, speech recognition technologies or motion recognition technologies may be used.

The input unit 150 according to embodiments of the present disclosure may be implemented as external input equipment connected to the user terminal 100. For example, the input equipment may be at least one of a touch pad, a touch pen, a keyboard, or a mouse for receiving the user input, but this is just an example and the present disclosure is not limited thereto.

The input unit 150 according to an embodiment of the present disclosure may recognize user touch input. The input unit 150 according to an embodiment of the present disclosure may be the same component as the output unit 140. The input unit 150 may be configured as a touch screen implemented to receive an input of the user. The touch screen may adopt any one scheme of a contact type capacitive scheme, an infrared light detection scheme, a surface ultrasonic wave (SAW) scheme, a piezoelectric scheme, and a resistance film scheme. A detailed description of the touch screen is just an example according to an embodiment of the present disclosure and various touch screen panels may be adopted in the user terminal 100. The input unit 150 configured as the touch screen may include a touch sensor. The touch sensor may be configured to convert a change in pressure applied to a specific portion of the input unit 150 or capacitance generated at the specific portion of the input unit 150 into an electrical input signal. The touch sensor may be configured to detect touch pressure as well as a touched position and area. When there is a touch input for the touch sensor, a signal(s) corresponding to the touch input is (are) sent to a touch controller. The touch controller processes the signal(s) and thereafter, transmits data corresponding thereto to the processor 120. As a result, the processor 120 may recognize which region of the input unit 150 is touched, and the like. Since the touch screen is an example of the input unit 150 assuming that the user terminal provides a graphic user interface, the input unit 150 is not limited to the above-described example and may be configured variously to suit the type of user interface.

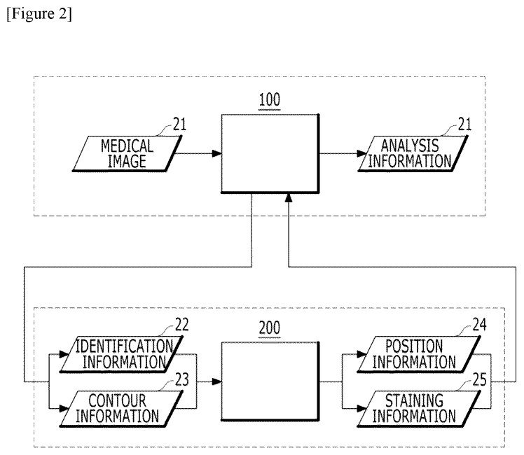

FIG. 2 is a block diagram illustrating a process for analyzing a medical image based on communication between a user terminal and a server according to an embodiment of the present disclosure.

Referring to FIG. 2, the user terminal 100 according to an embodiment of the present disclosure may determine a region of interest of a medical image 21 based on the medical image 21. At this time, the medical image 21 may be a pathology slide image in which cells stained through immunohistochemistry are expressed. For example, the medical image 21 may be a pathology slide image in which brown-colored cells are present due to ki-67 expression through DAB staining. Since the DAB staining-based pathology slide image is only one example of the medical image 21, the medical image 21 may include all various pathology slide images generated based on the immunohistochemistry.

The user terminal 100 may generate information on the region of interest of the medical image 21 based on the first user input on the user interface. The information on the region of interest of the medical image 21 may include identification information 22 of the region of interest and contour information 23 of the region of interest. The identification information 22 of the region of interest is information corresponding to an identification (ID) of the region of interest, and may be understood as unique information that distinguishes one region of interest present in the medical image from another region of interest. That is, the user terminal 100 may designate the region of interest in the medical image 21 according to the first user input, and generate the identification information 22 for distinguishing the region of interest and the contour information 23 for determining a region designated as the region of interest.

For example, the user terminal 100 may determine a region of interest to be analyzed in the medical image 21 based on a first user input for defining a point on the medical image 21 output through the user interface. When N (N is a natural number) points are defined in the medical image 21 by the first user input, the user terminal 100 may generate a closed contour connecting all of the N points, and determine a region having the closed contour as the contour as the region of interest in the medical image 21. Since a form of the closed contour (i.e., a form of the region of interest) may vary depending on how the N points are defined, the region of interest may be generated in various shapes including a circle or a polygon. When the region of interest is determined, the user terminal 100 may assign unique identification information 22 to the region of interest. Further, the user terminal 100 may generate contour information 23 of the region of interest based on the closed contour connecting all of the N points. In this case, the contour information 23 may include coordinate values of the N points constituting the closed contour. That is, the contour information 23 may be defined according to the first user input, and may include coordinate values of a point that becomes a reference of the contour of the region of interest.

Meanwhile, the user terminal 100 should be able to recognize that the first user input for determining the region of interest is completed. Accordingly, when the user terminal 100 recognizes at least one of an input for re-selecting a first defined point on the contour of the region of interest or an input for continuously selecting a last defined point on the contour of the region of interest, the user terminal 100 may consider that the first user input is complete and perform a calculation for determining the region of interest in the medical image 21. That is, the first user input may include at least one of an input for re-selecting the first defined point on the contour of the region of interest or an input of continuously selecting the last defined point on the contour of the region of interest, which corresponds to an input for making the contour of the region of interest into the closed contour.

For example, it is assumed that there is a state in which a first point, a second point, . . . , an Nth point for determining the contour of the region of interest are defined in the medical image 21 by the first user input. In this case, when an additional first user input for re-designating the first point is delivered to the user terminal 100 through the user interface, the user terminal 100 may recognize the additional first user input for re-designating the first point as an initiation signal of a calculation operation for determining the region of interest. Further, even when an additional first user input for continuously designating N points is delivered to the user terminal 100 through the user interface at the time of defining the N-th point, the user terminal 100 may recognize the additional first user input for continuously designating the N-th point as the initiation signal for the calculation operation for determining the region of interest. The user terminal 100 performs an operation according to the above-described initiation signal to generate the closed contour connecting all N points and determine the region having the closed contour as the contour as the region of interest in the medical image 21.

According to an embodiment of the present disclosure, the user terminal 100 may transmit the information on the region of interest in the medical image 21 to the server 200 that analyzes the stained cells present in the medical image. At this time, the server 200 may be understood as a computing system that transmits and receives information through communication with the user terminal 100 and performs analysis on the medical image. For example, the server 200 may receive the information on the region of interest in the medical image 21 from the user terminal 100, and recognize and count the stained cells present in the region of interest. The server 200 may deliver a result of recognizing and counting the stained cells present in the region of interest to the user terminal 100. That is, the server 200 may be understood as serving to perform analysis on the stained cells present in the region of interest through data communication with the user terminal 100, and provide an analysis result to the user terminal 100.

In order to enable efficient analysis by minimizing resources required for the data communication, the user terminal 100 may transmit information on a region of interest of the medical image 21 other than the entirety of the medical image 21 in which the region of interest is displayed or a part of the medical image 21 in which a region corresponding to the region of interest is extracted to the server 200. A medical image 21, such as a pathology slide image, is high-volume data ranging from hundreds of megabytes to several gigabytes, so when the user terminal 100 transmits the medical image 21 itself to the server 200, a large number of resources may be required during a data communication process. Additionally, when the user terminal 100 transmits the medical image 21 itself to the server 200, there is always a risk that various problems on a network and a memory may be caused. On the other hand, since the information on the region of interest including the identification information 22 and the contour information 23 is considerably low-volume data compared to the medical image 21, the data itself transmitted from the user terminal 100 to the server 200 becomes light, which may enable efficient data communication. In particular, when there are a plurality of regions of interest determined in the medical image 21, a capacity of transmitted data between the user terminal 100 and the server 200 is significantly reduced compared to the case of transmitting the medical image 21 itself, thereby maximizing the efficiency of the data communication.

According to an embodiment of the present disclosure, the server 200 may receive the identification information 22 and the contour information 23 of the region of interest from the user terminal 100 and generate positional information 24 and staining information 25 of the stained cells present in the region of interest of the medical image 21. The server 200 may extract an image corresponding to the region of interest from the medical image 21 based on the identification information 22 and contour information 23 of the region of interest received from the user terminal 100. At this time, the medical image 21 may be understood as data not delivered to the server 200 through the user terminal 100, but as data that the server 200 itself secures through communication with a database or external system. The server 200 may input the image of the region of interest generated based on the identification information 22 and the contour information 23 into a neural network model to generate the position information 24 of the stained cells present in the region of interest. The server 200 may generate the staining information 25 indicating a staining ratio of the stained cells based on a cell region of the region of interest corresponding to the position information 24 of the stained cells. At this time, the cell region may be a partial region of the region of interest surrounding the cell.

For example, the server 200 may input the image of the region of interest generated based on the identification information 22 and the contour information 23 into the neural network model to generate a bounding box corresponding to the cell region of the region of interest. At this time, the bounding box may be a polygonal image of a minimum size surrounding the stained cells. The server 200 may generate the bounding box through the neural network model and simultaneously obtain coordinate values of the bounding box as the position information 24 of the stained cells. The neural network model used in the server 200 may be pre-trained based on whether cells are positive or negative expressed through staining and a medical image in which the bounding box including cells is labeled.

The server 200 may estimate the staining ratio of stained cells in the cell region as the staining information 25 based on the bounding box. The server 200 may generate a binary image for the cell region based on a staining intensity of the bounding box. The server 200 may generate a binary image of the cell region surrounding the stained cells by comparing a staining expression intensity of the stained cells present in the bounding box with a predetermined threshold. At this time, the threshold predetermined in the server 200 may be the staining expression intensity that serves as a reference for distinguishing the stained cells as the positive cells. The server 200 may calculate the staining ratio of the stained cells in the cell region based on the binary image of the bounding box described above. At this time, the staining ratio may be understood as a ratio of an area of the positive region of cells expressed by staining within the cell region of the region of interest corresponding to a coordinate value to a total area of the cell region. In other words, the staining ratio may be understood as a ratio of positive cells expressed by staining to the entire region of the bounding box. Through the binary image, the cell region is clearly divided into a positive region and the remaining region according to the threshold, so the server 200 may accurately calculate the staining ratio of positive cells expressed by staining within the cell region by using the binary image.

According to an embodiment of the present disclosure, the user terminal 100 may distinguish cells present in the region of interest into positive or negative based on the position information 24 and the staining information 25 calculated through the neural network model of the server 200. Upon receiving the position information 24 and the staining information 25 of the stained cells within the region of interest from the server, the user terminal 100 may identify the stained cells within the region of interest through the position information 24 and identify a ratio of the stained cells present in the region of interest by using the identified staining information 25 of the staining cells. The user terminal 100 may distinguish the stained cells present in the region of interest as positive or negative based on a threshold that may be adjusted according to a second user input on the user interface based on the staining ratio of the stained cells whose position is identified. Further, the user terminal 100 may distinguish the positive cells present in the region of interest based on a threshold that may be adjusted according to the second user input on the user interface based on the staining ratio of the stained cells whose position is identified. At this time, the threshold, which may be adjusted according to the second user input, may include a first reference value indicating the staining expression intensity for distinguishing the cells present in the region of interest as positive or negative, or a second reference value for distinguishing the positive cells determined according to the first reference value according to the staining expression intensity. That is, the user terminal 100 may determine the stained cell as the positive cell or the negative cell by comparing the staining expression intensity of the stained cell of which position is identified in the region of interest with the first reference value of the threshold determined by the second user input. Further, the user terminal 100 may classify the positive cells into a plurality of classes according to the staining expression intensity by comparing the staining expression intensity of the positive cells determined through the first reference value with the second reference value of the threshold determined by the second user input. In other words, the user terminal 100 may perform positive-negative binary classification for the stained cells present in the region of interest using a threshold that includes at least one of the first reference value and the second reference value, and perform multi classification according to the staining expression intensity based on the positive cells.

For example, when the stained cells in the region of interest are displayed in a brown color through DAB staining, the user terminal 100 may identify brown cells in the region of interest based on the position information 24 of the brown-stained cells delivered from the server 200. Further, the user terminal 100 may determine a staining ratio of the identified brown cells in the cell region based on the staining information 25 delivered from the server 200. In this case, the cell region may be understood as a smallest region in the medical image containing the brown cells. The user terminal 100 compares the first reference value which may be adjusted according to the second user input with the staining ratio of the brown cells in the cell region to distinguish the brown cells which are equal to or more than the first reference value as DAB positive and brown cells which are equal to or less than the first reference value as DAB negative. Further, the user terminal 100 compares a staining ratio of the cells determined as the DAB positive in the cell region and a plurality of second reference values to distinguish the cells determined as the DAB positive into a plurality of classes according to the staining expression intensity in the cell region.

Meanwhile, when a threshold is adjusted by the second user input on the user interface, the user terminal 100 may update a result of determining whether the stained cell is positive or negative based on the threshold adjusted by the second user input. Since the user terminal 100 autonomously analyzes and distinguishes whether the stained cells are positive or negative, the user terminal 100 may filter whether the stained cells in the region of interest are positive or negative based on a threshold adjusted in real time without a need of additional communication with the server 200 even though the threshold is adjusted by the second user input. In other words, the user terminal 100 may autonomously control the threshold through the second user input delivered through the user interface, and distinguish the cell present in the region of interest as positive or negative by reflecting a change in threshold in real time. For example, the user terminal 100 may update the result of distinguishing the cells as positive or negative by again comparing the threshold changed by the second user input with the staining ratio of the stained cell within the cell region. At this time, as the threshold increases, the number of positive cells in the region of interest may decrease and the number of negative cells may increase. As the threshold decreases, the number of positive cells in the region of interest may increase and the number of negative cells may decrease.

The user terminal 100 may generate analysis information on stained cells in the region of interest based on the cells distinguished as positive or negative within the region of interest. The user terminal 100 counts positive cells and negative cells in the region of interest distinguished based on the threshold to generate analysis information including at least one of the numbers of positive cells and negative cells, respectively, and a first ratio based on the number of positive cells, or a second ratio based on an overall average of straining expression intensities of the stained cells present in the region of interest. For example, when it is assumed that the total number of stained cells identified in the region of interest is 10, the user terminal 100 counts the positive cells and the negative cells distinguished in the region of interest to determine the number of positive cells as 3 and the number of negative cells as 7, and calculate the numbers as one of the analysis information. Further, the user terminal 100 may calculate 0.3 (or 30%) which is a ratio occupied by 3 positive cells among a total of 10 stained cells based on the number of cells as one of the analysis information. The user terminal 100 may also calculate an average of staining expression intensities of a total of 10 stained cells as one of the analysis information based on the staining expression intensity of the stained cell in the region of interest. In this case, each of the ratio or the average of the straining expression intensity calculated as the analysis information may also be converted into a score according to a table stored in the memory 130. Specific numerical values relating to the above-described number or ratio are merely examples and the present disclosure is not limited thereto.

The user terminal 100 may output the analysis information related to the stained cell in the region of interest through the user interface. The user terminal 100 may display the stained cells in the medical image with different colors according to positive or negative so as for the user to easily and quickly determine the result of distinguishing the stained cell as positive or negative. Further, the user terminal 100 may display the negative cell and the positive cell with different colors, and distinguish the positive cells with different colors according to the staining expression intensity, and display the positive cells in the medical image. The user terminal 100 may visualize, through the user interface, a medical image in which both positive and negative cells for each class are displayed in different colors according to the staining expression intensity in the region of interest. The user terminal 100 may visualize at least one of the numbers of positive cells and negative cells, respectively included in the analysis information or statistical information related to the positive cells through the user interface. For example, the user terminal 100 may display cells distinguished as positive cells in red and cells distinguished as negative cells in blue among the stained cells in the region of interest in the medical image, and output the cells to a first region of the user interface. In addition, the user terminal 100 may classify the positive cells into 3 classes according to the staining expression intensity in the region of interest, and display respective classes in pink, orange, and red on the medical image and output the classes to the first region of the user interface. The user terminal 100 may visualize statistical information in the stained cell in the region of interest as text form information jointly with the numbers of positive cells and negative cells in the region of interest, and output the statistical information to a second region of the user interface. At this time, the statistical information of the stained cells in the region of interest may include at least one of a first ratio in which a ratio of the number of positive cells among all cells in the region of interest is converted into the score or a second ratio in which an average value of the staining expression intensities of all cells in the region of interest is converted into a score.

As such, the user terminal 100 calculates and visualizes the analysis information for the stained cell in the region of interest through communication of the server 200 to improve a delay problem of a pathology diagnosis test, and effectively assist judgment of a domain expert for a pathological state.

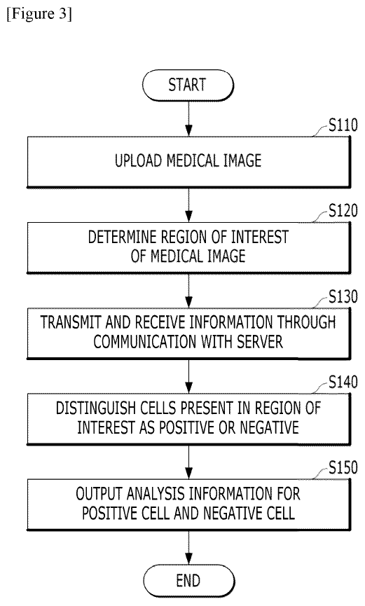

FIG. 3 is a flowchart illustrating a process of analyzing a medical image of a user terminal according to an embodiment of the present disclosure.

Referring to FIG. 3, in step S110, the user terminal 100 according to an embodiment of the present disclosure may upload a medical image through communication with a system that stores a medical image, such as PACS or an external database. The medical image may be a whole slide image (WSI) including cells stained through immunohistochemistry (e.g., DAB staining). At this time, the medical image such as the whole slide image is high-capacity data of hundreds of megabytes to several gigabytes, so when the medical image is uploaded and output as it is, the user terminal 100 will not smoothly reflect user's manipulation through the user interface to data. Accordingly, the user terminal 100 solves the above-mentioned problem by converting and storing a high-capacity medical image in advance and providing the image converted according to the user's manipulation through the user interface. For example, the user terminal 100 may convert medical images to be analyzed for each resolution level and store the medical images. At this time, the medical image converted for each resolution level may be a deep zoom image in which the image is organized into tiles according to the resolution level. The user terminal 100 may output, through the user interface, a medical image with a resolution level corresponding to a magnification level adjusted according to the user input on the user interface from the medical image stored after conversion for each resolution level. At this time, the user input can be understood as an input that adjusts the zoom level of a screen on which the medical image is to be output. That is, the user terminal 100 may output images of a region visible in the medical image through the user interface according to the magnification level adjusted according to the user input. Such medical image processing may effectively improve computational delay problems of the processor 120 of the user terminal 100 that may occur in the process of uploading high-capacity medical images and improve performance.

In step S120, the user terminal 100 may determine the region of interest of the medical image uploaded through step S110 according to the user input on the user interface. The user terminal 100 may generate ID information and contour position information of the region of interest that enable the server 200 to recognize the region of interest for efficient data communication. At this time, the region of interest in the medical image may be understood as a target region that the user wants to analyze in the whole slide image. The region of interest in the whole slide image may include cells and surrounding tissues stained through immunohistochemistry, which become targets for analysis.

In step S130, the user terminal 100 may transmit the ID information and the contour position information of the region of interest generated in step S120 to the server 200. Since only necessary information about the region of interest, not the medical image, is transmitted to the server 200, communication between the user terminal 100 and the server 200 may be performed smoothly without any network delay problem. The server 200 may easily identify the region of interest in the medical image based on the ID information and contour position information of the region of interest delivered from the user terminal 100. The server 200 may estimate positions of stained cells within the region of interest through the neural network model based on the region of interest. Additionally, the server 200 may calculate a staining ratio of positive cells in a specific region based on the staining expression intensities of the stained cells in the region of interest. Here, the specific region may be understood as a bounding box surrounding the stained cell corresponding to the position of the stained cell estimated through the neural network model. When the calculation of the server 200 is completed, the user terminal 100 may receive information on the positions of the stained cells and the ratio of positive cells in the region of interest generated by the server 200 from the server. Since only necessary information about the region of interest required for identifying the stained cell, not the medical image, is delivered to the user terminal 100, the communication between the user terminal 100 and the server 200 may be performed smoothly without any network delay problem.

In step S140, the user terminal 100 may distinguish whether the cells present in the region of interest are positive or negative based on the information on the stained cells delivered from the server 200 through step $130. The user terminal 100 may identify the stained cell present in the region of interest based on the information on the positions of the stained cells delivered from the server 200. The user terminal 100 compares the staining intensity of the stained cells of which positions are identified with the threshold determined by the user input on the user interface based on the information on the staining ratio of the positive cells delivered from the server 200 to distinguish the stained cells as the positive cells or the negative cells.

In step S150, the user terminal 100 may generate analysis information for the stained cells by counting the stained cells distinguished as positive or negative in the region of interest. The user terminal 100 may output the analysis information related to the stained cell in the region of interest through the user interface. The analysis information may include a medical image in which the positive and negative cells are displayed in different colors, the number of stained cells, and the ratio of positive cells. The user terminal 100 may visualize the analysis information related to the stained cell in the region of interest into at least one of the image or the text through the user interface. At this time, when a change occurs in the screen on which the region of interest is output according to user's manipulation through the user interface, the user terminal 100 does not re-perform a visualization task of displaying the positive cells and negative cells in different colors according to the changed screen. For an efficient operation, the user terminal 100 may perform a visualization task of displaying the positive and negative cells in different colors, image the region of interest, and then convert the imaged region of interest according to the user's manipulation and output the image which fits the changed screen. For example, the user terminal 100 may initially display positive and negative cells present in the region of interest in different colors and then convert the region of interest itself into a separate image. When the magnification level for the region of interest is adjusted by the user input on the user interface or the region of interest is moved in one region of the user interface, the user terminal 100 may convert a size or a coordinate of a region of interest converted into a separate image according to the adjusted magnification level or region to which the region of interest is moved, and output the size or coordinate through the user interface. Such medical image processing may effectively improve computational delay problems of the processor 120 of the user terminal 100 that may occur in the process of visualizing the information on the analyzed region of interest according to the user input, and improve performance.

Meanwhile, when there are a plurality of regions of interest, the user terminal 100 may organize information on the plurality of regions of interest into a list and transmit the information to the server 200. The server 200 may sequentially perform analysis on the regions of interest based on the list received from the user terminal 100. The server 200 may collect the information on the analyzed region of interest and transmit the information to the user terminal 100. The user terminal 100 may selectively output individual analysis information for the regions of interest, aggregation analysis information for each group of the regions of interest, analysis information acquired by aggregating all regions of interest, etc., according to conditions set according to user input on the user interface.

FIGS. 4 to 6 are conceptual views exemplarily illustrating a user interface according to an embodiment of the present disclosure.

Referring to FIG. 4, the user interface according to an embodiment of the present disclosure may include a first region 31 that outputs a medical image initially uploaded to determine a region of interest or a medical image in which the region of interest is visualized. The region of interest may be generated, modified, or changed according to a user input which is input through the first region 31 of the user interface.

According to an embodiment of the present disclosure, when a medical image that is a target for determining the region of interest is output through the first region 31, the user may designate a region of interest which wants to analyze in the medical image through the user input for the first region 31. The user may deliver an input for defining a point of the region of interest to the user terminal 100 through the first region 31. The user terminal 100 may determine various types of regions of interest such as a square region of interest \textcircled{a} and a heptagonal region of interest \textcircled{b} according to the input through the first region 31. At this time, the user terminal 100 may judge whether a crossing line or an overlapping region exists between the region of interest \textcircled{a} and the region of interest \textcircled{b}, and determine the contour of the region of interest so that no crossing line or overlapping region occurs. The user terminal 100 may immediately visualize the square region of interest \textcircled{a} and the heptagonal region of interest \textcircled{b}, display the regions in the medical image, and then output the regions through the first region 31.

Referring to FIG. 5, the user interface according to an embodiment of the present disclosure may include a second region 32 that outputs an analysis result for the region of interest. The analysis result for the region of interest may include the numbers of positive cells and negative cells present in the region of interest, respectively, the ratio of positive cells based on the region of interest, etc. Information output through the second region 32 as information analyzed by the user terminal 100 may not be basically changed, modified, or adjusted by the user input. However, depending on a setting value of the user interface, the information output through the second region 32 may also be changed, modified, or adjusted by the user input. Additionally, the user interface may include a third region 33 that outputs information on a first reference value indicating a staining expression intensity, which is a judgment reference of the positive cell in the region of interest. The first reference value may be adjusted according to user input which is input through the third region 33 of the user interface.

According to an embodiment of the present disclosure, after the region of interest is defined in the medical image through the first region 31, the user may control the user terminal 100 to analyze the region of interest through inputting an analyze icon displayed in the third region 33. At this time, the first reference value may be displayed in the third region 33 as a numerical value in a text form and a bar-shaped icon in a graphic form. The user may adjust the first reference value displayed in the third region 33 before, after, or in the middle of analyzing the region of interest of the user terminal 100. The first reference value adjusted by the user input for the third region 33 may be reflected in real time to the analysis of the region of interest of the user terminal 100.

When the analysis of the region of interest is completed by the user terminal 100, the user terminal 100 may output a medical image with the positive and negative cells displayed in different colors through the first region 31. At the same time, the user terminal 100 may output the numbers of positive cells and negative cells in the region of interest and the ratio of positive cells in the region of interest through the second region 32. Information output through the second region 32 may be displayed as the numerical value in a text form. For example, when the medical image is a pathology slide image of cells stained with a DAB solution, the user terminal 100 may judge a brown expression intensity of the stained cells in the region of interest according to the first reference value displayed in the third region 33, and classify the stained cells into the positive cells or the negative cells. Based on a classification result, the user terminal 100 may display and output the positive cells in red and the negative cells in blue in the region of interest through the first region 31. Additionally, the user terminal 100 may count positive cells and negative cells based on the classification result, calculate the number of stained cells and the ratio of positive cells, and output the number and the ratio through the second region 32. After checking the analysis result output through the user interface, the user may terminate the operation of the user terminal 100 by performing a user input on a save icon or report icon displayed in the second region 32.

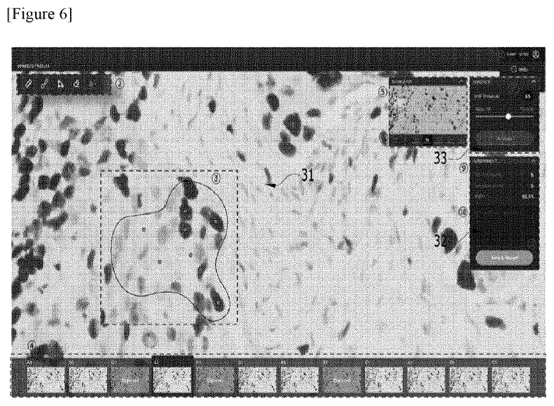

FIG. 6 illustrates an example of a whole screen output from the user terminal 100 according to an embodiment of the present disclosure. Functions of user interface elements implemented on the screen may be organized as shown in [Table 1] below. Each user interface element may be implemented on the screen by the user input or a calculation operation by the user terminal 100. The screen displayed in FIG. 6 is just an example and the present disclosure is not limited thereto.

TABLE 1

| Classification | Functions |
|---|---|
| \textcircled{1} | Set-up interface that returns a folder to upload the medical image to an |
| | in initial state to be re-designated according to the user input |
| \textcircled{2} | Tool interface that enables designation and deletion of a region to be |
| | analyzed by the user, length measurement, and color change |
| \textcircled{3} | Interface that displays a region of interest designated in free shape and |
| | size according to an intention of the user |
| \textcircled{4} | List interface that displays an image list in the folder designated |
| | through \textcircled{1} and allows an image selected according to the user input to |
| | be shown on the screen |
| \textcircled{5} | Navigator interface, as a mini map of a whole image, which displays a |
| | position (coordinate) of a region which is being currently analyzed to |
| | be checked by the user. |
| \textcircled{6} | Magnification interface that enables the user to set an enlargement |
| | magnification of the image |
| \textcircled{7} | Threshold interface that enables the user to adjust a color of a staining |
| | slide and a staining intensity reference which are judgment references |
| | of positive cells |
| \textcircled{8} | Analyze interface which allows the user terminal to perform analysis |
| | by applying the reference set through \textcircled{7} by the user |
| \textcircled{9} | Assessment interface that displays a counting result and a ratio of |
| | positive cells and negative cells in the region of interest analyzed by |
| | the user terminal |
| \textcircled{10} | Comment interface that enables the user to write an additional opinion |
| | for the analysis result of the user terminal |
| \textcircled{11} | Interface that enables the result analyzed by the user terminal to be |
| | stored and a report for the result to be output |
| \textcircled{12} | Menu interface that displays a user manual, a logout icon, etc. |

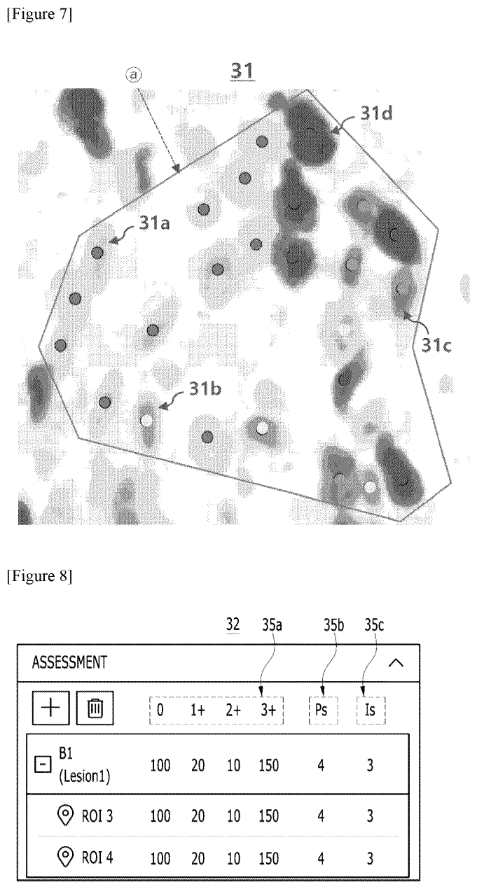

FIGS. 7 to 9 are conceptual views exemplarily illustrating a user interface according to an additional embodiment of the present disclosure. FIGS. 4 to 6 are conceptual views exemplarily illustrating a user interface that displays a result of binary classification that distinguishes stained cells as negative or positive cells, while FIGS. 7 to 9 may be understood as conceptual views exemplarily illustrating a user interface that displays results of multiple classifications that additionally distinguish positive cells into a plurality of classes according to the staining expression intensity. Regarding FIGS. 7 to 9, contents that overlap with the previous description of FIGS. 4 to 6 will be omitted below. Additionally, since specific numerical values described in relation to FIGS. 7 to 9 are merely examples, the present disclosure is not limited thereto. The user terminal 100 according to an additional embodiment of the present disclosure may display and output stained cells in the region of interest in different colors according to the class through the first region 31 of the user interface. Referring to FIG. 7, the stained cells present in the region of interest \textcircled{a} of the first region 31 may be classified into negative cell 31a, positive cells 1 31b, positive cells 2 31c, and positive cells 3 31d. First, the stained cells present in the region of interest \textcircled{a} of the first region 31 may be classified into the negative cell 31a, or the positive cells 31b, 31c, and 31d according to the first reference value. In addition, the stained cells classified as the positive cells 31b, 31c, and 31d may be classified into one of three classes classified based on the staining expression intensity according to the second reference value. The stained cells 31a, 31b, 31c, and 31d present in the region of interest \textcircled{a} of the first region 31 classified into one of four classes may be displayed in different colors. The negative cell 31a may be displayed in blue, positive cell 1 31b may be displayed in pink, positive cell 2 31c may be displayed in orange, and positive cell 3 31d may be displayed in red. The user terminal 100 according to an additional embodiment of the present disclosure may output not only counting information of stained cells in the region of interest but also statistical information of stained cells converted into a score in the text form through the second region 32 of the user interface. For example, referring to FIGS. 7 and 8, the user terminal 100 may output the number of stained cells for each of four classes (0, 1+, 2+, and 3+) for each region of interest (32a), a ratio of positive cells within the region of interest (32b), and an average staining intensity within the region of interest (32c) through the second region 32. The four classes (0, 1+, 2+, and 3+) may correspond to the negative cell 31a, positive cell 1 31b, positive cell 2 31c, and positive cell 3 31d, respectively. Specifically, class 0 may correspond to the negative cell 31a determined according to the first reference value. Class 1+ may correspond to positive cell 1 31b determined according to the first and second reference values. Class 2+ may correspond to positive cell 2 31c determined according to the first and second reference values. Class 3+ may correspond to positive cell 3 31d determined according to the first and second reference values. At this time, this may mean that the staining expression intensities of the positive cell increase from class 1 toward class 3+.

The ratio 32b of the positive cells in the region of interest may be a score calculated based on the number of positive cells present in a specific region of interest. The user terminal 100 may calculate a ratio of positive cells to all stained cells present in the region of interest. The user terminal 100 may convert the ratio of the positive cells into a score according to a table stored in the memory 130 and output the ratio 32b of the positive cells in the region of interest through the second region 32. At this time, the ratio 32b of the positive cells in the region of interest may be a score corresponding to one of the natural numbers between 0 and 5. A score closer to 5 may mean a higher ratio of the positive cells within the region of interest.

The average staining intensity 32c within the region of interest may be a score calculated based on the staining expression intensities of staining cells present within a specific region of interest. The user terminal 100 may calculate an average value of staining expression intensities of all stained cells present in the region of interest. The user terminal 100 may convert the calculated average value into a score according to the table stored in the memory 130 and output the average staining intensity 32c in the region of interest through the second region 32. At this time, the ratio 32b of the positive cells in the region of interest may be a score corresponding to one of the natural numbers between 0 and 3. Specifically, when an overall average value of the staining expression intensities within the region of interest is 0.5 or less, the user terminal 100 may calculate 0 as the average staining intensity 32c within the region of interest. When the overall average value of the staining expression intensities within the region of interest is more than 0.5 and 0.67 or less, the user terminal 100 may calculate 1 as the average staining intensity 32c within the region of interest. When the overall average value of the staining expression intensities within the region of interest is more than 0.67 and 0.83 or less, the user terminal 100 may calculate 2 as the average staining intensity 32c within the region of interest. When the overall average value of the staining expression intensities within the region of interest is more than 0.83 and 1 or less, the user terminal 100 may calculate 3 as the average staining intensity 32c within the region of interest. Accordingly, a score closer to 3 may mean a higher ratio of the positive cells within the region of interest.

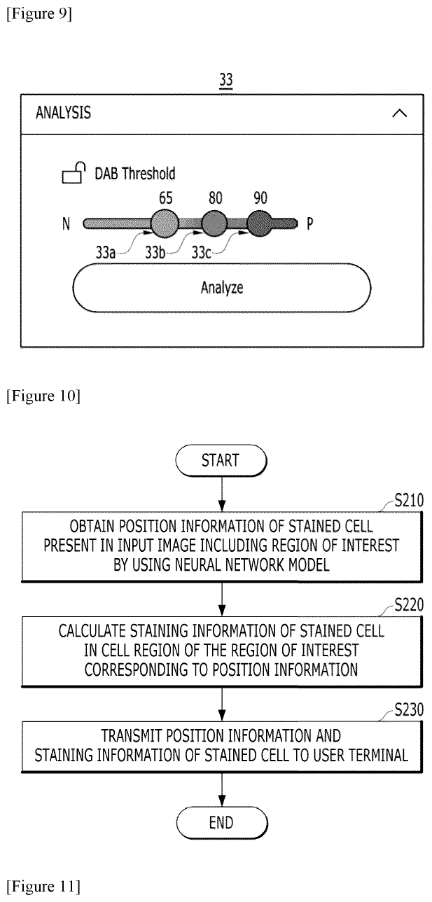

Referring to FIG. 9, the user interface according to an additional embodiment of the present disclosure may include a third region 33 that outputs information on a first reference value 33a indicating a staining expression intensity which becomes a judgment reference of positive cells in a region of interest, and second reference values 33b and 33c which become classification references for straining expression intensities of the positive cells. The first reference value 33a and the second reference values 33b and 33c may be adjusted according to user input which is input through the third region 33 of the user interface. For example, the first reference value 33a and the second reference values 33b and 33c may be displayed in the third region 33 as a numerical value in a text form and a bar-shaped icon in a graphic form, respectively. The user may adjust the first reference value 33a and the second reference values 33b and 33c displayed in the third region 33 before, after, or in the middle of analyzing the region of interest of the user terminal 100. The first reference value 33a and the second reference values 33b and 33c adjusted by the user input for the third region 33 may be reflected in real time to the analysis of the region of interest of the user terminal 100. A result analyzed based on the first reference value 33a and the second reference value 33b and 33c displayed in the third region 33 may be displayed as in the first region 31 of FIG. 7 in which the stained cells are displayed in different colors on the medical image. Further, a result analyzed based on the first reference value 33a and the second reference value 33b and 33c displayed in the third region 33 may be displayed as the numerical value in the text form, as in the second region 32 of FIG. 8.

FIG. 10 is a flowchart illustrating an operation process of a server according to an embodiment of the present disclosure.

Referring to FIG. 10, in step S210, the server 200 according to an embodiment of the present disclosure may receive information about the region of interest through communication with the user terminal 100. At this time, the information on the region of interest may be understood as unique information of a region designated as the region of interest by the user in a whole slide image (WSI) including cells stained through immunohistochemistry (e.g., DAB staining). The server 200 may extract an image of the region of interest from the whole slide image (WSI) based on the unique information of the region of interest delivered from the user terminal 100. In addition, the server 200 may obtain position information of stained cells present in the medical image using a neural network model. For example, the server 200 inputs the image of the region of interest extracted from the whole slide image (WSI) into the neural network model to obtain coordinate values of the stained cells present in the region of interest. At this time, the server 200 may obtain a bounding box containing the stained cells along with the coordinate values of the stained cells through the neural network model. At this time, the bounding box may be a polygonal or circular image of a minimum size adjacent to contours of the stained cells.

In step S220, the server 200 may extract an image of a predetermined region including the stained cells from the image of the region of interest based on the position information of the stained cells calculated in step S210. For example, the server 200 may estimate a bounding box corresponding to the coordinate value of the stained cell based on the coordinate value of the stained cell, and extract an image corresponding to the bounding box from the image of the region of interest. At this time, when the bounding box is obtained along with the coordinate value of the stained cell in step S210, the server 200 may not extract the image of the predetermined region including the stained cell from the image of the region of interest, but use the bounding box obtained in step S210 as it is. In step S220, the server 200 may calculate the staining ratio of stained cells within one region of the image of the region of interest corresponding to the position information calculated in step S210. At this time, one region of the image of the region of interest may be a bounding box region previously extracted from the medical image. The server 200 may compare a staining intensity with a threshold and calculate a staining ratio indicating how large the positive region of cells expressed by staining occupies based on a whole region of the bounding box.

In step S230, the server 200 may transmit the position information of the stained cells calculated through step S210 and the staining ratio calculated through step S220 to the user terminal 100. The server 200 may transmit, to the user terminal 100, only information requisitely utilized for pathological diagnosis, rather than the analyzed image itself. Through this, the server 200 prevents an unnecessarily large amount of resources from being required in the process of transmitting and receiving data with the user terminal 100 and minimizes costs to enable efficient data communication. The position information of the stained cells calculated by the server 200 in step S210 and the staining ratio of the stained cells calculated by the server 200 in step S220 may be effectively used in a calculation process of counting the stained cells for the pathological diagnosis. In addition, the position information of the stained cells calculated by the server 200 in step S210 and the staining ratio of the stained cells calculated by the server 200 in step S220 may be effectively used for visualizing the image for the stained cells and the counting result of the stained cells through the user terminal 100.

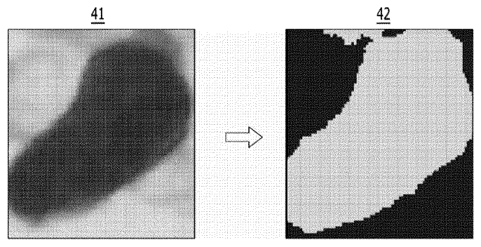

FIG. 11 is a conceptual view of a process of generating a binary image of the server according to an embodiment of the present disclosure.

The processor of the server 200 according to an embodiment of the present disclosure may generate a bounding box image 41 surrounding DAB-stained cells present in the region of interest in the medical image using a pre-trained neural network model. Since the bounding box image 41 is a minimum size image that includes all regions of one DAB-stained cell, the bounding box image 41 inevitably includes not only the DAB-stained cell but also some surrounding tissues of the DAB-stained cell. The bounding box image 41 in FIG. 4 is a square image, but the bounding box image 41 may be another multi-angled image such as a pentagonal image, or a circular image depending on setting by the user.

In order to accurately and easily calculate the staining ratio of the DAB-stained cells present in the bounding box image 41, the processor of the server 200 may convert the bounding box image 41 into a binary image 42. In order to convert the bounding box image 41 into the binary image 42, the processor of the server 200 may binary-process the bounding box image 41 by comparing staining intensities of pixels in the bounding box image 41 with a threshold corresponding to a minimum reference intensity to be classified into a DAB positive cell. That is, the processor of the server 200 may generate the binary image 42 based on pixels corresponding to positive cells expressed by DAB staining in the bounding box image 41. In the binary image 42, a gray region indicates the DAB positive region and a black region indicates the DAB negative region or a surrounding tissue region other than the DAB stained cell region. The processor of the server 200 easily distinguishes the DAB positive region through the binary image 42 to quickly and accurately calculate a ratio of an area of the DAB positive region to a whole region of the binary image 42.

In the meantime, according to an embodiment of the present disclosure, a computer readable medium storing a data structure is disclosed.

The data structure may refer to organization, management, and storage of data that enable efficient access and modification of data. The data structure may refer to organization of data for solving a specific problem (for example, data search, data storage, and data modification in the shortest time). The data structure may also be defined with a physical or logical relationship between the data elements designed to support a specific data processing function. A logical relationship between data elements may include a connection relationship between user defined data elements. A physical relationship between data elements may include an actual relationship between the data elements physically stored in a computer readable storage medium (for example, a permanent storage device). In particular, the data structure may include a set of data, a relationship between data, and a function or a command applicable to data. Through the effectively designed data structure, the computing device may perform a calculation while minimally using resources of the computing device. In particular, the computing device may improve efficiency of calculation, reading, insertion, deletion, comparison, exchange, and search through the effectively designed data structure.

The data structure may be divided into a linear data structure and a non-linear data structure according to the form of the data structure. The linear data structure may be the structure in which only one data is connected after one data. The linear data structure may include a list, a stack, a queue, and a deque. The list may mean a series of dataset in which order exists internally. The list may include a linked list. The linked list may have a data structure in which data is connected in a method in which each data has a pointer and is linked in a single line. In the linked list, the pointer may include information about the connection with the next or previous data. The linked list may be expressed as a single linked list, a double linked list, and a circular linked list according to the form. The stack may have a data listing structure with limited access to data. The stack may have a linear data structure that may process (for example, insert or delete) data only at one end of the data structure. The data stored in the stack may have a data structure (Last In First Out, LIFO) in which the later the data enters, the sooner the data comes out. The queue is a data listing structure with limited access to data, and may have a data structure (First In First Out, FIFO) in which the later the data is stored, the later the data comes out, unlike the stack. The deque may have a data structure that may process data at both ends of the data structure.

The non-linear data structure may be the structure in which the plurality of data is connected after one data. The non-linear data structure may include a graph data structure. The graph data structure may be defined with a vertex and an edge, and the edge may include a line connecting two different vertexes. The graph data structure may include a tree data structure. The tree data structure may be the data structure in which a path connecting two different vertexes among the plurality of vertexes included in the tree is one. That is, the tree data structure may be the data structure in which a loop is not formed in the graph data structure.

Throughout the present specification, a calculation model, a nerve network, the network function, and the neural network may be used with the same meaning. Hereinafter, the terms of the calculation model, the nerve network, the network function, and the neural network are unified and described with a neural network. The data structure may include a neural network. Further, the data structure including the neural network may be stored in a computer readable medium. The data structure including the neural network may also include preprocessed data for processing by the neural network, data input to the neural network, a weight of the neural network, a hyper-parameter of the neural network, data obtained from the neural network, an active function associated with each node or layer of the neural network, and a loss function for training of the neural network. The data structure including the neural network may include predetermined configuration elements among the disclosed configurations. That is, the data structure including the neural network may include the entirety or a predetermined combination of pre-processed data for processing by neural network, data input to the neural network, a weight of the neural network, a hyper parameter of the neural network, data obtained from the neural network, an active function associated with each node or layer of the neural network, and a loss function for training the neural network. In addition to the foregoing configurations, the data structure including the neural network may include predetermined other information determining a characteristic of the neural network. Further, the data structure may include all type of data used or generated in a computation process of the neural network, and is not limited to the foregoing matter. The computer readable medium may include a computer readable recording medium and/or a computer readable transmission medium. The neural network may be formed of a set of interconnected calculation units which are generally referred to as “nodes.” The “nodes” may also be called “neurons.” The neural network consists of one or more nodes.

The data structure may include data input to the neural network. The data structure including the data input to the neural network may be stored in the computer readable medium. The data input to the neural network may include training data input in the training process of the neural network and/or input data input to the training completed neural network. The data input to the neural network may include data that has undergone pre-processing and/or data to be pre-processed. The pre-processing may include a data processing process for inputting data to the neural network. Accordingly, the data structure may include data to be pre-processed and data generated by the pre-processing. The foregoing data structure is merely an example, and the present disclosure is not limited thereto.

The data structure may include a weight of the neural network (in the present specification, weights and parameters may be used with the same meaning), Further, the data structure including the weight of the neural network may be stored in the computer readable medium. The neural network may include a plurality of weights. The weight is variable, and in order for the neural network to perform a desired function, the weight may be varied by a user or an algorithm. For example, when one or more input nodes are connected to one output node by links, respectively, the output node may determine a data value output from the output node based on values input to the input nodes connected to the output node and the weight set in the link corresponding to each of the input nodes. The foregoing data structure is merely an example, and the present disclosure is not limited thereto.

For a non-limited example, the weight may include a weight varied in the neural network training process and/or the weight when the training of the neural network is completed. The weight varied in the neural network training process may include a weight at a time at which a training cycle starts and/or a weight varied during a training cycle. The weight when the training of the neural network is completed may include a weight of the neural network completing the training cycle. Accordingly, the data structure including the weight of the neural network may include the data structure including the weight varied in the neural network training process and/or the weight when the training of the neural network is completed. Accordingly, it is assumed that the weight and/or a combination of the respective weights are included in the data structure including the weight of the neural network. The foregoing data structure is merely an example, and the present disclosure is not limited thereto.

The data structure including the weight of the neural network may be stored in the computer readable storage medium (for example, a memory and a hard disk) after undergoing a serialization process. The serialization may be the process of storing the data structure in the same or different computing devices and converting the data structure into a form that may be reconstructed and used later. The computing device may serialize the data structure and transceive the data through a network. The serialized data structure including the weight of the neural network may be reconstructed in the same or different computing devices through deserialization. The data structure including the weight of the neural network is not limited to the serialization. Further, the data structure including the weight of the neural network may include a data structure (for example, in the non-linear data structure, B-Tree, Trie, m-way search tree, AVL tree, and Red-Black Tree) for improving efficiency of the calculation while minimally using the resources of the computing device. The foregoing matter is merely an example, and the present disclosure is not limited thereto.

The data structure may include a hyper-parameter of the neural network. The data structure including the hyper-parameter of the neural network may be stored in the computer readable medium. The hyper-parameter may be a variable varied by a user. The hyper-parameter may include, for example, a learning rate, a cost function, the number of times of repetition of the training cycle, weight initialization (for example, setting of a range of a weight value to be weight-initialized), and the number of hidden units (for example, the number of hidden layers and the number of nodes of the hidden layer). The foregoing data structure is merely an example, and the present disclosure is not limited thereto.

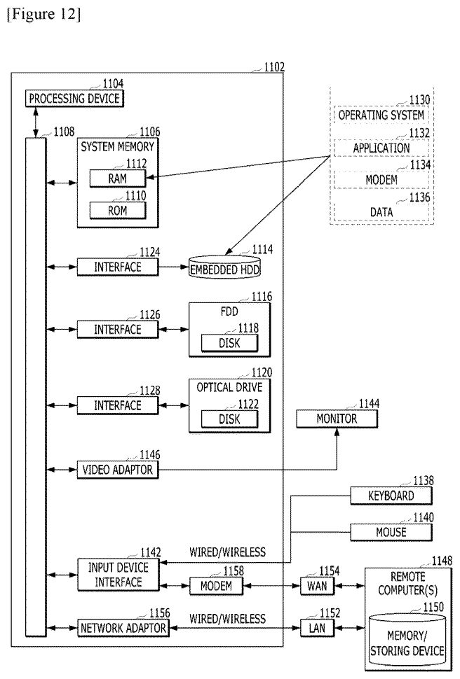

FIG. 12 is a simple and general schematic diagram illustrating an example of a computing environment in which the embodiments of the present disclosure are implementable.

The present disclosure has been described as being generally implementable by the computing device, but those skilled in the art will appreciate well that the present disclosure is combined with computer executable commands and/or other program modules executable in one or more computers and/or be implemented by a combination of hardware and software.

In general, a program module includes a routine, a program, a component, a data structure, and the like performing a specific task or implementing a specific abstract data form. Further, those skilled in the art will well appreciate that the method of the present disclosure may be carried out by a personal computer, a hand-held computing device, a microprocessor-based or programmable home appliance (each of which may be connected with one or more relevant devices and be operated), and other computer system configurations, as well as a single-processor or multiprocessor computer system, a mini computer, and a main frame computer,

The embodiments of the present disclosure may be carried out in a distribution computing environment, in which certain tasks are performed by remote processing devices connected through a communication network. In the distribution computing environment, a program module may be located in both a local memory storage device and a remote memory storage device.

The computer generally includes various computer readable media. The computer accessible medium may be any type of computer readable medium, and the computer readable medium includes volatile and non-volatile media, transitory and non-transitory media, and portable and non-portable media. As a non-limited example, the computer readable medium may include a computer readable storage medium and a computer readable transport medium. The computer readable storage medium includes volatile and non-volatile media, transitory and non-transitory media, and portable and non-portable media constructed by a predetermined method or technology, which stores information, such as a computer readable command, a data structure, a program module, or other data. The computer readable storage medium includes a RAM, a Read Only Memory (ROM), an Electrically Erasable and Programmable ROM (EEPROM), a flash memory, or other memory technologies, a Compact Disc (CD)-ROM, a Digital Video Disk (DVD), or other optical disk storage devices, a magnetic cassette, a magnetic tape, a magnetic disk storage device, or other magnetic storage device, or other predetermined media, which are accessible by a computer and are used for storing desired information, but is not limited thereto.

The computer readable transport medium generally implements a computer readable command, a data structure, a program module, or other data in a modulated data signal, such as a carrier wave or other transport mechanisms, and includes all of the information transport media. The modulated data signal means a signal, of which one or more of the characteristics are set or changed so as to encode information within the signal. As a non-limited example, the computer readable transport medium includes a wired medium, such as a wired network or a direct-wired connection, and a wireless medium, such as sound, Radio Frequency (RF), infrared rays, and other wireless media. A combination of the predetermined media among the foregoing media is also included in a range of the computer readable transport medium.

An illustrative environment 1100 including a computer 1102 and implementing several aspects of the present disclosure is illustrated, and the computer 1102 includes a processing device 1104, a system memory 1106, and a system bus 1108. The system bus 1108 connects system components including the system memory 1106 (not limited) to the processing device 1104. The processing device 1104 may be a predetermined processor among various commonly used processors. A dual processor and other multi-processor architectures may also be used as the processing device 1104.

The system bus 1108 may be a predetermined one among several types of bas structure, which may be additionally connectable to a local bus using a predetermined one among a memory bus, a peripheral device bus, and various common bus architectures. The system memory 1106 includes a ROM 1110, and a RAM 1112. A basic input/output system (BIOS) is stored in a non-volatile memory 1110, such as a ROM, an EPROM, and an EEPROM, and the BIOS includes a basic routing helping a transport of information among the constituent elements within the computer 1102 at a time, such as starting. The RAM 1112 may also include a high-rate RAM, such as a static RAM, for caching data.

The computer 1102 also includes an embedded hard disk drive (HDD) 1114 (for example, enhanced integrated drive electronics (EIDE) and serial advanced technology attachment (SATA))-the embedded HDD 1114 being configured for exterior mounted usage within a proper chassis (not illustrated)-a magnetic floppy disk drive (FDD) 1116 (for example, which is for reading data from a portable diskette 1118 or recording data in the portable diskette 1118), and an optical disk drive 1120 (for example, which is for reading a CD-ROM disk 1122, or reading data from other high-capacity optical media, such as a DVD, or recording data in the high-capacity optical media). A hard disk drive 1114, a magnetic disk drive 1116, and an optical disk drive 1120 may be connected to a system bus 1108 by a hard disk drive interface 1124, a magnetic disk drive interface 1126, and an optical drive interface 1128, respectively. An interface 1124 for implementing an outer mounted drive includes, for example, at least one of or both a universal serial bus (USB) and the Institute of Electrical and Electronics Engineers (IEEE) 1394 interface technology.

The drives and the computer readable media associated with the drives provide non-volatile storage of data, data structures, computer executable commands, and the like. In the case of the computer 1102, the drive and the medium correspond to the storage of random data in an appropriate digital form. In the description of the computer readable media, the HDD, the portable magnetic disk, and the portable optical media, such as a CD, or a DVD, are mentioned, but those skilled in the art will well appreciate that other types of computer readable media, such as a zip drive, a magnetic cassette, a flash memory card, and a cartridge, may also be used in the illustrative operation environment, and the predetermined medium may include computer executable commands for performing the methods of the present disclosure.

A plurality of program modules including an operation system 1130, one or more application programs 1132, other program modules 1134, and program data 1136 may be stored in the drive and the RAM 1112. An entirety or a part of the operation system, the application, the module, and/or data may also be cached in the RAM 1112. It will be well appreciated that the present disclosure may be implemented by several commercially usable operation systems or a combination of operation systems.

A user may input a command and information to the computer 1102 through one or more wired/wireless input devices, for example, a keyboard 1138 and a pointing device, such as a mouse 1140. Other input devices (not illustrated) may be a microphone, an IR remote controller, a joystick, a game pad, a stylus pen, a touch screen, and the like. The foregoing and other input devices are frequently connected to the processing device 1104 through an input device interface 1142 connected to the system bus 1108, but may be connected by other interfaces, such as a parallel port, an IEEE 1394 serial port, a game port, a USB port, an IR interface, and other interfaces.

A monitor 1144 or other types of display devices are also connected to the system bus 1108 through an interface, such as a video adaptor 1146. In addition to the monitor 1144, the computer generally includes other peripheral output devices (not illustrated), such as a speaker and a printer.

The computer 1102 may be operated in a networked environment by using a logical connection to one or more remote computers, such as remote computer(s) 1148, through wired and/or wireless communication. The remote computer(s) 1148 may be a work station, a computing device computer, a router, a personal computer, a portable computer, a microprocessor-based entertainment device, a peer device, and other general network nodes, and generally includes some or an entirety of the constituent elements described for the computer 1102, but only a memory storage device 1150 is illustrated for simplicity. The illustrated logical connection includes a wired/wireless connection to a local area network (LAN) 1152 and/or a larger network, for example, a wide area network (WAN) 1154. The LAN and WAN networking environments are general in an office and a company, and make an enterprise-wide computer network, such as an Intranet, easy, and all of the LAN and WAN networking environments may be connected to a worldwide computer network, for example, the Internet.

When the computer 1102 is used in the LAN networking environment, the computer 1102 is connected to the local network 1152 through a wired and/or wireless communication network interface or an adaptor 1156. The adaptor 1156 may make wired or wireless communication to the LAN 1152 easy, and the LAN 1152 also includes a wireless access point installed therein for the communication with the wireless adaptor 1156. When the computer 1102 is used in the WAN networking environment, the computer 1102 may include a modem 1158, is connected to a communication computing device on a WAN 1154, or includes other means setting communication through the WAN 1154 via the Internet. The modem 1158, which may be an embedded or outer-mounted and wired or wireless device, is connected to the system bus 1108 through a serial port interface 1142. In the networked environment, the program modules described for the computer 1102 or some of the program modules may be stored in a remote memory/storage device 1150. The illustrated network connection is illustrative, and those skilled in the art will appreciate well that other means setting a communication link between the computers may be used.

The computer 1102 performs an operation of communicating with a predetermined wireless device or entity, for example, a printer, a scanner, a desktop and/or portable computer, a portable data assistant (PDA), a communication satellite, predetermined equipment or place related to a wirelessly detectable tag, and a telephone, which is disposed by wireless communication and is operated. The operation includes a wireless fidelity (Wi-Fi) and Bluetooth wireless technology at least. Accordingly, the communication may have a pre-defined structure, such as a network in the related art, or may be simply ad hoc communication between at least two devices.

The Wi-Fi enables a connection to the Internet and the like even without a wire. The Wi-Fi is a wireless technology, such as a cellular phone, which enables the device, for example, the computer, to transmit and receive data indoors and outdoors, that is, in any place within a communication range of a base station. A Wi-Fi network uses a wireless technology, which is called IEEE 802.11 (a, b, g, etc.) for providing a safe, reliable, and high-rate wireless connection. The Wi-Fi may be used for connecting the computer to the computer, the Internet, and the wired network (IEEE 802.3 or Ethernet is used). The Wi-Fi network may be operated at, for example, a data rate of 11 Mbps (802.11a) or 54 Mbps (802.11b) in an unauthorized 2.4 and 5 GHz wireless band, or may be operated in a product including both bands (dual bands).

Those skilled in the art may appreciate that information and signals may be expressed by using predetermined various different technologies and techniques. For example, data, indications, commands, information, signals, bits, symbols, and chips referable in the foregoing description may be expressed with voltages, currents, electromagnetic waves, magnetic fields or particles, optical fields or particles, or a predetermined combination thereof.

Those skilled in the art will appreciate that the various illustrative logical blocks, modules, processors, means, circuits, and algorithm operations described in relationship to the embodiments disclosed herein may be implemented by electronic hardware (for convenience, called “software” herein), various forms of program or design code, or a combination thereof. In order to clearly describe compatibility of the hardware and the software, various illustrative components, blocks, modules, circuits, and operations are generally illustrated above in relation to the functions of the hardware and the software. Whether the function is implemented as hardware or software depends on design limits given to a specific application or an entire system. Those skilled in the art may perform the function described by various schemes for each specific application, but it shall not be construed that the determinations of the performance depart from the scope of the present disclosure.

Various embodiments presented herein may be implemented by a method, a device, or a manufactured article using a standard programming and/or engineering technology. A term “manufactured article” includes a computer program, a carrier, or a medium accessible from a predetermined computer-readable storage device. For example, the computer-readable storage medium includes a magnetic storage device (for example, a hard disk, a floppy disk, and a magnetic strip), an optical disk (for example, a CD and a DVD), a smart card, and a flash memory device (for example, an EEPROM, a card, a stick, and a key drive), but is not limited thereto. Further, various storage media presented herein include one or more devices and/or other machine-readable media for storing information.

It shall be understood that a specific order or a hierarchical structure of the operations included in the presented processes is an example of illustrative accesses. It shall be understood that a specific order or a hierarchical structure of the operations included in the processes may be rearranged within the scope of the present disclosure based on design priorities. The accompanying method claims provide various operations of elements in a sample order, but it does not mean that the claims are limited to the presented specific order or hierarchical structure.

The description of the presented embodiments is provided so as for those skilled in the art to use or carry out the present disclosure. Various modifications of the embodiments may be apparent to those skilled in the art, and general principles defined herein may be applied to other embodiments without departing from the scope of the present disclosure. Accordingly, the present disclosure is not limited to the embodiments suggested herein, and shall be interpreted within the broadest meaning range consistent to the principles and new characteristics presented herein.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for analyzing a medical image performed by a user terminal including at least one processor, the method comprising:
determining a region of interest of a medical image based on a first user input on a user interface;
transmitting identification information and contour information of the region of interest to a server, and receiving position information and staining information of a stained cell present in the region of interest from the server;
distinguishing the cell present in the region of interest as positive or negative based on a threshold which is adjustable according to a second user input on the user interface by using the position information and the staining information of the stained cell present in the region of interest; and
outputting analysis information for the cell distinguished as a positive or negative through the user interface.

2. The method of claim 1, further comprising:
converting and storing a medical image to be analyzed for each resolution level; and
outputting a medical image of a resolution level corresponding to a magnification level adjusted according to a third user input on the user interface through the user interface, in the stored medical image.

3. The method of claim 1, wherein the determining of the region of interest of the medical image based on the first user input on the user interface includes:
generating the region of interest of the medical image as a closed contour according to the first user input.

4. The method of claim 1, wherein the contour information of the region of interest includes a coordinate value of a point which becomes a reference of a contour of the region of interest, and
wherein the point which becomes the reference of the contour of the region of interest is determined according to the first user input.

5. The method of claim 1, wherein the position information of the stained cell present in the region of interest includes: a coordinate value of the stained cell present in the region of interest, which is calculated from an input image including the region of interest through a neural network model of the server, and
wherein the staining information of the stained cell present in the region of interest includes: a staining ratio of the stained cell, which is calculated in a cell region of the region of interest corresponding to the coordinate value by the server.

6. The method of claim 5, wherein the staining ratio of the stained cell is a ratio of an area of a positive region of the cell expressed by staining in the cell region to a total area of the cell region.

7. The method of claim 5, wherein the staining ratio of the stained cell is calculated based on a binary image for the cell region generated based on a staining intensity of the region of interest by the server.

8. The method of claim 1, wherein the threshold adjustable according to the second user input on the user interface includes a first reference value indicating a staining expression intensity for distinguishing the cell present in the region of interest into positive or negative.

9. The method of claim 8, wherein the threshold adjustable according to the second user input on the user interface further includes a second reference value for distinguishing a positive cell determined according to the first reference value according to a staining expression intensity.

10. The method of claim 1, wherein the distinguishing of the cell present in the region of interest as positive or negative includes:
comparing the threshold and the staining information of the stained cell present in the region of interest and distinguishing the cell as positive or negative.

11. The method of claim 10, wherein the comparing of the threshold and the staining information of the stained cell present in the region of interest and distinguishing of the cell as positive or negative includes:
comparing a first reference value included in the threshold and the staining information of the stained cell present in the region of interest, and distinguishing the cell as positive or negative, and
comparing a second reference value included in the threshold and staining information of a positive cell distinguished according to the first reference value, and distinguishing the positive cell according to a staining expression intensity.

12. The method of claim 1, wherein the distinguishing of the cell present in the region of interest as positive or negative includes:
updating, when the threshold is adjusted by the second user input on the user interface, a result of distinguishing the cell as positive or negative based on the adjusted threshold.

13. The method of claim 1, wherein the outputting of the analysis information through the user interface includes:
outputting a medical image in which a positive cell and a negative cell distinguished by using a first reference value included in the threshold are displayed in different colors to a first region of the user interface.

14. The method of claim 13, wherein positive cells distinguished by using the first reference value included in the threshold are displayed in different colors according to a staining expression intensity distinguished by using a second reference value included in the threshold.

15. The method of claim 13, wherein the outputting of the medical image in which the positive cell and the negative cell distinguished by using the threshold are displayed in different colors to the first region of the user interface includes:
storing a region of interest of the medical image in which the positive cell and the negative cell distinguished by using the threshold are displayed in different colors as a separate image, and
converting, when a magnification level for the region of interest is adjusted or the region of interest is moved from the first region according to a third user input on the user interface, a size or a coordinate of the region of interest stored as the separate image according to the third user input.

16. The method of claim 1, wherein the outputting of the analysis information through the user interface includes:
outputting, to a second region of the user interface, at least one of a number of negative cells distinguished by using the threshold, a number of positive cells distinguished by using the threshold, a first ratio based on the number of positive cells, or a second ratio based on an overall average of staining expression intensities of stained cells present in the region of interest.

17. The method of claim 1, wherein the user interface includes:
a first region receiving the first user input, and outputting a medical image for determining the region of interest, a medical image in which the region of interest is visualized, or a medical image in which a positive cell and a negative cell distinguished by using the threshold displayed in different colors,
a second region outputting at least one of a number of negative cells distinguished by using the threshold, a number of positive cells distinguished by using the threshold, a first ratio based on the number of positive cells, or a second ratio based on the overall average of staining expression intensities of stained cells present in the region of interest, and
a third region receiving the second user input, and outputting information on the threshold.

18. A computer program stored in a computer-readable storage medium, wherein when the computer program is executed by one or more processors, the computer program cause the one or more processor to execute following operations for analyzing a medical image, the operations comprising:
an operation of determining a region of interest of a medical image based on a first user input on a user interface;
an operation of distinguishing a cell present in the region of interest as positive or negative based on a threshold which is adjustable according to a second user input on the user interface by using position information and staining information of a stained cell present in the region of interest; and
an operation of outputting analysis information for the cell distinguished as positive or negative through the user interface,
wherein the position information and the staining information of the stained cell present in the region of interest is received from a server receiving identification information and contour information of the region of interest.

19. A user terminal for analyzing a medical image, comprising:
a processor comprising one or more cores;
a memory;
a network unit for communication with a server; and
an output unit providing a user interface,
wherein the processor is configured to:
determine a region of interest of a medical image based on a first user input on a user interface,
distinguish a cell present in the region of interest as positive or negative based on a threshold which is adjustable according to a second user input on the user interface by using position information and staining information of a stained cell present in the region of interest, and
output analysis information for the cell distinguished as positive or negative through the user interface, and
the network unit transmits identification information and contour information of the region of interest to the server, and receives the position information and the staining information of the stained cell present in the region of interest from the server.
